(19) United States
(12) Patent Application Publication
(10) Pub. No.: US 2020/0078199 A1
(43) Pub. Date: Mar. 12, 2020

(54) LEG CORRECTING APPARATUS

(71) Applicants: Dong Ho SONG, Seoul (KR); Jinwook SONG, Jung-gu Daegu (KR)

(72) Inventors: Dong Ho SONG, Seoul (KR); Jinwook SONG, Daegu (KR); Joon Han KANG, Seoul (KR)

(21) Appl. No.: 16/493,141

(22) PCT Filed: Apr. 6, 2018
(86) PCT No.: PCT/KR2018/004096

§ 371 Date: Sep. 11, 2019

(30) Foreign Application Priority Data
Apr. 14, 2017 (KR) 10-2017-0048295

(51) Int. Cl. A61F 5/01; A61F 5/34

(52) U.S. Cl. CPC A61F 5/012; A61F 5/0125; A61F 2005/0179; A61F 2005/0172; A61F 5/34

(57) ABSTRACT

The present invention relates to a leg correcting apparatus and, more particularly, to a leg correcting apparatus capable of correcting leg curvature such as genu varum (bow legs) or genu valgum (knock knees) caused by tibial lateral torsion.

### TECHNICAL FIELD

The present invention relates to a leg correcting apparatus and, more particularly, to a leg correcting apparatus capable of correcting leg curvature such as genu varum (bow legs) or genu valgum (knock knees) caused by tibial lateral torsion.

### BACKGROUND ART

The present invention relates to a leg correcting apparatus and to an apparatus which corrects genu varum (bow legs) or genu valgum (knock knees) caused by tibial lateral torsion.

The patella can move along the femral groove without applying stress on the joint faces when its internal side or external side moves in a balance of force, and thus failure to balance the knee joint increases stress, patella permanent pain syndrome (PFPS), and a problem may occur around the patella portion, and the knee joint is made up of many ligaments than the other joints and serves as a support.

The knee joint is a joint between the lower end of the femur and the upper end of the tibia, and the back surface of the patella, i.e., the bones of the three parts, and due to a variety of reasons including rickets, osteomalacia, cartilage growth disorder, endocrine abnormalities, bent posture, imbalanced diet, malnutrition, and lack of exercise, abnormality may occur in the knee joint, and legs may be crooked inwardly or outwardly which is referred to as crooked legs including genu varum and genu valgum.

More specifically, the knee joint is a place where inner and outer sides of the femur lower end and inner and outer sides of the tibia upper end, and a castle dismisses joint where the knee flexion and extension are made by forming a hinge joint with the patella slit attached to the front. It is a hinge joint between the medial malleolus and tibia, and between the lateral malleolus and tibia, and formed with a plane joint between the patella and the femur. The knee joint is surrounded by bursitis full of synovial fluid, which absorbs friction from impact and protects the joints.

In the knee joint, a tibio-femoral angle appears, which is an angle at which the long axis of the femur meets the long axis of the tibia vertically, and which forms between 185 degree and 195 degree inside the knee and thus the femoral axis deviates by 5 to 10 degree from the long axis of the tibia. This is called a physiological valgus, and if it is greater than normal valgus, it is generally referred to as genu valgum (knock knees) which causes the external side of the knee joint to be under pressure.

Further, a form of the tibio-femoral angle smaller than normal valgus is referred to as genu varum (bow legs) which causes the internal side of the knee joint to be under pressure.

That is, at an internal side of the knee, the long axis line of the femur and the long axis line of the tibia meet at right angles, and the change in this angle determines genu varum and genu valgum, and is an angle of body weight carried by the knee so the deformation of the knee causes an internal or an external lateral deviation of the knee.

In a case where no measure is taken for genu varum or genu valgum, all of them can cause secondary diseases, which can have a negative impact on health, and thus treatment through correction is necessary.

As a prior art related to an treatment apparatus for such genu varum or genu valgum, Korean patent application publication No. 2005-0110567 (Nov. 23, 2005) is directed to a leg correcting apparatus, and more specifically, discloses a technology relating to an leg correcting apparatus including and constituted with a main body mounted on an internal side of a user's leg and supported on the user's knee at one end and on the user's ankle at the other end, and a pressurizing means that is connected to both sides of the middle part of the main body to wrap the crooked part of a leg so that the crooked leg can be corrected by applying force to the crooked part of the leg, and, Korean registered utility model No. 20-0236205 (Oct. 25, 2001) is directed to a crooked leg correcting apparatus, and more specifically, discloses a technology regarding a correcting device in which two cylinders are fixed by a fixing screw at both sides so as to become in a straight line, U-shaped front and rear sides of upper and lower cylinders allow for free knee flexion, and which is capable of tightening the crooked part, using a pressurizing screw.

Aforementioned Korean patent application publication No. 2005-0110567 and Korean Registered utility model No. 20-0236205, which correspond to prior art, describe technologies on devices that reversely pressurize and correct the crooked legs, using fixing means and pressurizing means, but the above technique, which is intended to pressurize and correct the crooked tibia part, could not remedy the crooked leg by correcting the tibial lateral torsion.

According to above described situations, the invention attempts to present an apparatus which corrects genu varum or genu valgum by correcting tibia lateral torsion so that the tibio-femoral angle of the knee joint become within the normal range.

### DISCLOSURE

### Technical Problem

The invention is to address above described problems, and aims to provide a leg correcting apparatus configured to more effectively correct genu varum or genu valgum caused by tibial external lateral torsion by applying pressure on the upper external side portion of the leg antebrachium in an internal side direction.

Further, the invention aims to provide a leg correcting apparatus which enables pressure applied to the knee portion to be adjusted easily according to the leg condition of a user, and at the same time whose wearing feeling is improved.

Further, it is still another object for the invention to provide a leg correcting apparatus which allows a knee portion to bent easily when a user wears it and walks or takes a rest, by making the knee position bendable, and whose correcting effect is further maximized by adding normal flexibility together with knee internal side correcting pressurizing.

### Technical Solution

The invention is characterized in that a leg collecting apparatus includes a support frame which can support a brachium and an antebrachium of a leg, and is formed along an up and down direction of the leg; a thigh fixing part which is connected to the support frame and installed so as to wrap and fix a thigh portion of the leg brachium; a calf fixing part which is connected to the support frame and installed so as to wrap and fix a calf portion of the leg antebrachium; and a knee fixing part which is located between the thigh fixing part and the calf fixing part, is connected to the support frame and installed so as to support at least a knee joint portion of the leg antebrachium side, wherein the knee fixing part includes a knee internal side support part which is connected and fixed to the support frame, and supports a knee internal side, and a knee external side support part which is connected to the support frame and supports the knee external side, and which can push the knee joint portion of the leg antebrachium side from the leg external side in the leg internal side direction to apply a pressure on the knee external side portion.

As an exemplary embodiment, the support frame may include an upper support frame supporting the leg brachium, and a lower support frame supporting the leg antebrachium, and wherein the upper support frame, the lower support frame or a coupling part thereof may include a knee bendable part which has a structure that can be bent according to a bending angle of a knee.

As an exemplary embodiment, the upper support frame or the lower support frame may be provided with one axis at a rear side of a leg, or provided with a pair of axes at the internal side and the external side of the leg brachium.

As an exemplary embodiment, the knee bendable part may be kept unbent by an elastic body including at least one or more selected from a spring, a wire and a rubber if no external force is applied thereto.

As an exemplary embodiment, the thigh fixing part and the calf fixing part may be configured such that their heights can be adjusted along a length direction of a leg when being installed to the support frame, or the support frame may be configured such that their lengths can be adjusted along an up and down length direction of a leg.

As an exemplary embodiment, the thigh fixing part may include a thigh internal side support part which is connected to the support frame and supports a thigh internal side of the leg brachium, and a thigh external side support part which is connected to the support frame and fastened with the thigh internal side support part to support a thigh external side; or the calf fixing part may include a calf internal side support part which is connected to the support frame and supports a calf portion internal side of the leg antebrachium, and a calf external side support part which is connected to the support frame and fastened with the calf internal side support part to support a calf external side.

As an exemplary embodiment, the thigh internal side support part, the knee internal side support part and the calf internal side support part in the leg correcting apparatus may be respectively fixed to the support frame, and may be configured to support an internal side of each of the thigh, the knee and the calf in response to a case where pressure is applied to the knee external side portion.

As an exemplary embodiment, a knee external side support part in the leg correcting apparatus may include an air cushion part which pressurizes the knee joint portion of the leg antebrachium side by pressure of injected air in the internal side direction in order to push the knee joint portion of the leg antebrachium from the leg external side in the leg internal side direction.

As an exemplary embodiment, the leg correcting apparatus may further include an air supplier as a separate apparatus injecting air to the air cushion part, wherein the air supplier includes a pressurizer injecting air to the air cushion part of the knee external side support part, and a tube through which air moves from the pressurizer to the air cushion part, an air pressure display part displaying air pressure of the air cushion part, and an air vent part for removing air pressure of the air cushion part.

As an exemplary embodiment, the leg correcting apparatus may further include an ankle fixing part which is connected to the support frame and installed so as to wrap and support an ankle portion of the leg antebrachium, wherein the ankle fixing part includes an ankle internal side support part which is connected to the support frame and wraps an ankle portion internal side of the leg antebrachium, and an ankle external side support part which is connected to the support frame and fastened with the ankle internal side support part to support the ankle external side.

Further, the invention provides a leg correcting apparatus including: a support frame which can support a brachium and an antebrachium of a leg, and is formed along an up and down direction of the leg; a thigh fixing part which is connected to the support frame and installed so as to wrap and fix a thigh portion of the leg brachium; a calf fixing part which is connected to the support frame and installed so as to wrap and fix a calf portion of the leg antebrachium; and a knee fixing part which is located between the thigh fixing part and the calf fixing part, is connected to the support frame and installed so as to support at least a knee joint portion of the leg antebrachium side, wherein the knee fixing part includes a knee external side support part which is connected and fixed to the support frame, and supports a knee external side, and a knee internal side support part which is connected to the support frame and supports the knee internal side, and which can push the knee joint portion of the leg antebrachium side from the leg internal side in the leg external side direction to apply a pressure on the knee internal side portion.

### Advantageous Effects

Advantageously, the leg correcting apparatus according to the invention can provide excellent leg correcting effect because the tibial externally and laterally deviated can be effectively corrected to a normal position due to a pressure applied in a specific direction only on the knee joint portion of a leg antebrachium side by pushing the knee joint portion of the leg antebrachium side from the leg external side in the leg internal side direction to apply pressure on the knee external side portion, and because the knee joint is added in a state in which pressure is applied to the knee external side.

Further, the leg correcting apparatus according to the invention has a additional effect that by pressurizing the knee joint portion of the leg antebrachium side by pressure of air injected only into the knee joint portion of the leg antebrachium side, the pressure applied to the knee portion can be adjusted more precisely and easily according to the user's leg condition, and at the same time, wearing feeling can be improved when wearing it.

### DESCRIPTION OF DRAWINGS

FIG. 1 is a drawing for explaining the configuration of a leg correcting apparatus according to an exemplary embodiment of the invention.

FIG. 2 is a drawing for explaining the configuration of a leg correcting apparatus according to another exemplary embodiment of the invention.

FIG. 3 is a drawing for explaining position adjustment of a leg fixing part in the leg correcting apparatus according to an exemplary embodiment of the invention.

FIG. 4 is a drawing for explaining a leg correcting apparatus including an ankle fixing part according to another exemplary embodiment of the invention.

FIG. 5 is a drawing for explaining an air cushion part of a knee fixing part in the leg correcting apparatus according to an exemplary embodiment of the invention.

FIG. 6 is a drawing for explaining an air pressure indicating part of the knee fixing part in the leg correcting apparatus according to an exemplary embodiment of the invention.

### REFERENCE SIGNS LIST

100: leg correcting apparatus

110: support frame

111: upper support frame

112: lower support frame

114: knee bendable part

120: leg fixing part

121: thigh fixing part

121-1: thigh inner supporting part

121-2: thigh outer supporting part

121-3: thigh fixing belt

122: knee fixing part

122-1: knee inner supporting part

122-2: knee outer supporting part

123: calf fixing part

123-1: calf inner supporting part

123-2: calf outer supporting part

123-3: calf fixing belt

124: air cushion part

125: air supplying part

126: air vent part

127: ankle fixing part

127-1: ankle inner supporting part

127-2: ankle outer supporting part

127-3: ankle fixing belt

130: air supplier

130-1: pressurizer

130-2: tube

130-3: air pressure display part

130-4: air vent part

### MODE FOR INVENTION

It should be noted that, when supplementing constitutional elements with reference numerals, for the like constitutional elements, even though appearing on a different drawing, it is intended to make them have like reference numerals as much as possible. Further, the terms, “first”, “second”, “a surface”, “the other surface”, and the like are used to distinguish one constitutional element from anther constitutional element, and, however, the constitutional elements are not limited by the aforementioned terms.

When describing in detail principles for a preferred embodiment of the present invention, if it is determined that specific description on a related known function or a configuration would unnecessarily obscure essential contents of the present invention, such description will be omitted.

Further, exemplary embodiments described in the present specification and configurations illustrated in the drawings are just preferred exemplary embodiments, and do not represent every technical idea of the present invention, and thus it should be understood that there might be, at a time of filing this application, various equivalents and modified examples which could replace them.

Hereinafter, a preferred exemplary embodiment of a leg correcting apparatus 100 according to the present invention will be described referring to the attached drawings in such a detail that the ordinarily skilled in the art can implement the present invention with ease.

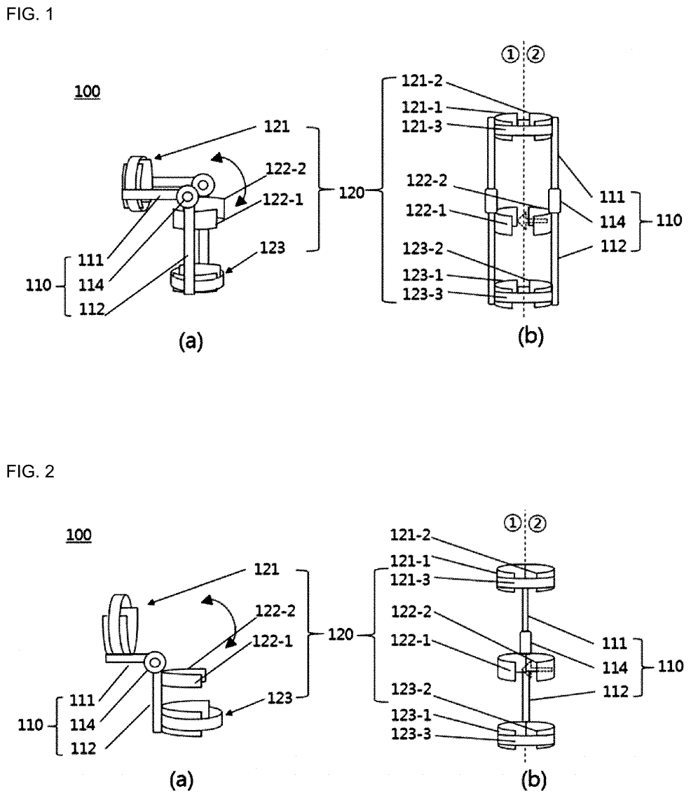

In the leg correcting apparatus 100 according to the present invention, FIG. 1 is showing a leg correcting apparatus 100 in which a support frame is provided with a pair of axes, one at an inner side of the leg brachium and the other at an outer side, and FIG. 2 is showing a leg correcting apparatus 100 in which a support frame is provided with one axis at a rear side of a leg.

Here, the leg correcting apparatus 100 shown in a right side of respective FIGS. 1 and 2 is intended to be described as a front surface of the drawings being worn on a front surface of a user's leg, a rear surface direction of the drawing being worn on a rear surface of the user's leg, and also as a side indicted by \textcircled{1} based on a vertical dotted line shown in the right side of FIGS. 1 and 2 being worn to be positioned at the inner side of the leg and a side indicted by \textcircled{2} being worn to be positioned at the outer side of the leg.

As shown in FIGS. 1 and 2, the leg correcting apparatus 100 according to an exemplary embodiment of the invention may include a support frame 110 and a leg fixing part (120), and the leg fixing part 120 may include a thigh fixing part 121, a knee fixing 122, and a calf fixing part (123).

In the support frame 110 of the present invention, the leg fixing part 120 may be installed, may support both a brachium and an antebrachium of the leg, and is formed along an up and down length direction of a leg.

Meanwhile, as used in the present invention, the “leg brachium” implies between a hip joint and a knee, the “leg antebrachium” implies between a knee and an ankle, and the support frame 110 in the present invention is preferably configured to include an upper support frame 111 supporting the leg brachium and a lower support frame 112 supporting the leg antebrachium, wherein the upper support frame 111 and the lower support frame 112 may be configured integrally or detachably.

Further, in the upper support frame 111, the lower support frame 112 or a coupling part thereof, provided is a knee bendable part 114 which has a structure that can be bent according to a bending angle of a knee, and thus when a user wears the leg correcting apparatus 100 of the present invention in order to use it, it can allow the user to perform a motion of bending or unbending the knee with ease.

That is, the knee bendable part 114 may be provided at a lower end of the upper support frame 111 or an upper end of the upper support frame 112, or so as to be positioned at the coupling part between the upper support frame 111 and the lower support frame 112, and in FIGS. 1 and 2, by way of an example, the one provided so as to be positioned at between the upper frame 111 and the lower support frame 112 is illustrated.

For example, even though the knee bendable part 114 may be provided at the lower end of the upper support frame 111 or the upper end of the upper support frame 112, the upper support frame 111 and the lower support frame 112 may be connected to each other by having a separate coupling part therebetween.

In the meantime, the knee bendable part 114 may be kept unbent by an elastic body including at least one or more selected from a spring, a wire and a rubber if no external force is applied thereto, so that, when a user wears the leg correcting apparatus 100 of the present invention and walk, it can help to walk.

That is, in a state where a user does not apply any force to the leg correcting apparatus 100 of the present invention, an initial state of the leg correcting apparatus 100 keeps the knee bendable part 114 unbent without being bent, so that it allows a user to wear with ease, and avoiding a need for a user to apply a unnecessary force when the user is in a standing position.

On the other hand, the leg correcting apparatus 100 may keep a bent state by keeping an initial state so as to maintain a predetermined angle in a state where no external force is applied, so that it can make it easy to wear in a sitting position while maintaining the bent state.

Meanwhile, in the present invention, the leg fixing part 120 is connected and installed in the support frame 110, and as described above, the leg fixing part 120 include the thigh fixing part 121, the knee fixing part 122, and the calf fixing part 123 according to a fixing point.

When specifically examining a particular configuration and function of each of the leg fixing part 120, the thigh fixing part 121 is connected to the support frame 110 and installed so as to wrap and fix a thigh portion of the leg brachium, the calf fixing part 123 is connected to the support frame 110 and installed so as to wrap and fix a calf portion of the leg antebrachium, and the knee fixing part 122 is located between the thigh fixing part 121 and the calf fixing part 123, connected to the support frame 110 and installed so as to support at least a knee joint portion of the leg antebrachium.

Meanwhile, the present invention is characterized in that the thigh fixing part 121, the knee fixing part 122 and the calf fixing part 123, which correspond to the leg fixing parts connected to the support frame 110, support the thigh portion of the leg brachium, the knee portion and the calf portion of the leg antebrachium, respectively, and the knee fixing part 122 applies pressure on a knee joint portion of the leg antebrachium from a leg external side portion in an internal side direction to push the tibial inward, thus correcting genu varum (bow legs).

For this, the knee fixing part 122 may include a knee internal side support part 122-1 which is connected and fixed to the support frame 110, and supports the knee internal side, and a knee external side support part 122-2 which is connected to the support frame 110 and supports the knee external side, and which can push the knee joint portion of the leg antebrachium from the leg external side in the internal side direction to apply a pressure on the knee external side portion.

That is, in the leg correcting apparatus 100 according to the present invention, the thigh fixing part, the knee fixing part and the calf fixing part are respectively fixed to the support frame 110, making the knee external side support part 122-2 to apply pressure on the knee joint external side portion of the antebrachium side, and in response to this, making each of the thigh fixing part, the knee fixing part and the calf fixing part to support the leg inside, thus correcting the tibial externally and laterally deviated to be moved into a normal position.

Further, as described above, the knee fixing part 122 should be located at a lower part that at least the knee bendable part 114 since it should be installed so as to push the knee joint external side portion of the leg antebrachium and apply pressure, while supporting the opposite knee side.

Therefore, the knee fixing part 122 may be connected to the lower support frame 112, or connected to the coupling part between the upper support frame 111 and the lower support frame 112.

Meanwhile, as shown in FIGS. 1 and 2, the support frame 110 of the leg correcting apparatus 100 according to the present invention may include the upper support frame or the lower support frame, and the upper support frame or the lower support frame may be configured with one axis or with two or more axes, and preferably provided with one axis at the rear side of a leg or with a pair of axes at the internal side and the external side of a leg brachium.

More specifically, FIG. 1 shows that the support frame 110 is provided with one axis at the rear side of a leg, FIG. 2 shows that the support frame 110 is provided with a pair of axes at the internal side and the external side of a leg, and the invention is not limited to this, and may be adjusted as various modifications such as one in which each of the upper support frame 111 and the lower support frame 112 is differently provided with one axis at the rear side of a leg or with a pair of axes at the internal side and the external side of a leg.

That is, the upper support frame 111 and the lower support frame 112 may be all provided with one axis at the rear side of a leg, or with a pair of axes at the internal side and the external side of a leg, or the upper support frame 111 may be provided with one axis at the rear side of a leg while the lower support frame 112 may be provided with a pair of axes at the internal side and the external side of a leg. The upper support frame 111 may be provided with a pair of axes at the internal side and the external side of a leg while the lower support frame 112 may be provided with one axis at the rear side of a leg.

Meanwhile, the height of the leg fixing part 120 according to the invention may be adjusted along the length direction.

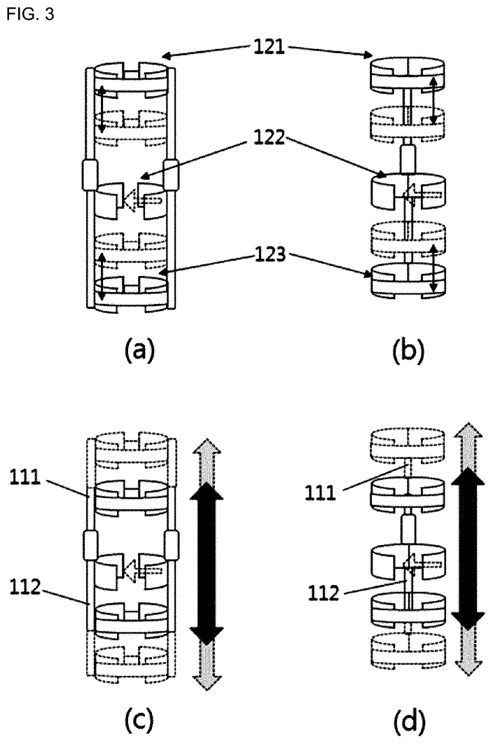

Referring to FIG. 3, which is an exemplary drawing for explaining about position adjustment of the leg fixing part 120 in the leg correcting apparatus 100, the thigh fixing part 121 and the calf fixing part 123 may be installed on the support frame 110, such that their height can be adjusted in the length direction of a leg to be worn correspondingly according to leg length of various user.

Further, the support frame 110 according to the invention may be configured such that its length can be adjusted along the up and down length direction. That is, as shown in FIGS. 3C and 3D, each of the upper support frame and lower support frame may be provided with height adjustable groove such as multiple coupling grooves in the interior of each support frame, may be allowed to adjust its length by bolt-nut or the like, and the coupling part of the upper support frame and the lower support frame may be provided with a fastening part which can adjust its length using a screw rod and a nut, or a sawtooth and a latch, whereby a user can be allowed to make length adjustment with ease.

Meanwhile, at least one or more leg fixing part 120 which is selected from among the thigh fixing part 121 or the calf fixing part 123 may include the internal support part supporting the internal side of a leg and the external support part supporting the external side of a leg.

More specifically, the thigh fixing part 121 may include a thigh internal side support part 121-1 which is connected to the support frame 110 and supports a thigh portion internal side of the leg brachium, and a thigh external side support part 121-2 which is connected to the support frame 110 and fastened with the thigh portion internal side support part 121-1 to support a thigh external side; or the calf fixing part 123 may include a calf internal side support part 123-1 which is connected to the support frame 110 and supports a calf portion internal side, and a calf external side support part 123-2 which is connected to the support frame 110 and fastened with the calf internal side support part 123-1 to support a calf external side.

Here, the thigh internal side support part 121-1 and the thigh external side support part 121-2 may be connected to each other at a knee front side surface and a knee rear side surface to be integrally configured, and similarly, the calf internal side support part 123-1 and the calf external side support part 123-2 may be connected to each other at the knee front side surface and the knee rear side surface to be integrally configured.

Further, the invention may include a thigh fixing belt 121-3 fastening the thigh internal side support part and the thigh external side support part, and in response to this, may include a calf fixing belt 123-3 fastening the calf internal support part and the calf external support part.

Here, the thigh internal side support part and the calf internal side support part are configured to support internal side surfaces of the thigh and the calf together with the knee internal side support part in response to the pressure from the knee external support part 122-2, so that a knee joint portion of the leg antebrachium side can be moved to a normal range to be corrected.

For this, in the leg correcting apparatus 100 of the invention, the thigh internal side support part 121-1, the knee internal side support part 122-1 and the calf internal side support part 123-1 in the leg correcting apparatus 100 may be respectively fixed to the support frame 110, and be configured to support the internal side of each of the thigh, the knee and the calf in response to a case where pressure is applied to the knee external side portion.

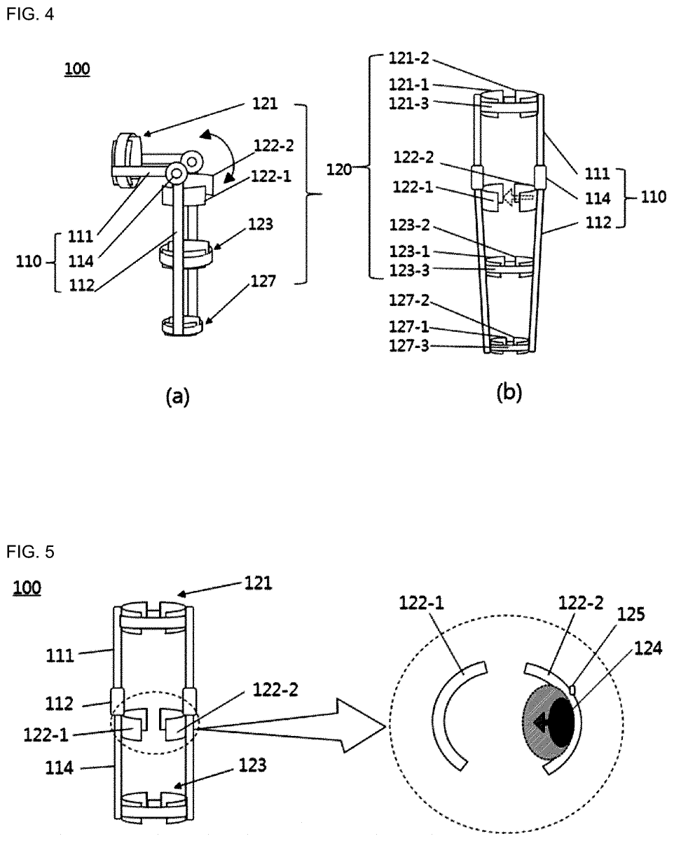

Meanwhile, the invention may be configured to include an ankle fixing part 127 at a lower portion of the calf fixing part as shown in FIG. 4 for improving the effect of correcting a leg by increasing a supporting force which supports the internal side surface of a leg in response to the pressure applied to the knee external side portion.

The ankle fixing part 127 may be connected to the support frame 110, and be installed so as to wrap and support the ankle portion of the leg antebrachium side, and the ankle fixing part 127 may include an internal side support part 127-1 which is connected to the support frame 110, and wraps the ankle portion of a leg antebrachium, and an external side support part 127-2 which is connected to the support frame 110, and fastened with the ankle internal side support part 127-1 to support the ankle external side.

Here, the ankle internal side support part 127-1 and the ankle external side support part 127-2 may be connected to each other at the knee front side surface or the knee rear side surface to be integrally configured similarly to the above, and may include an ankle fixing belt 127-3 fastening the ankle internal side support part and the ankle external side support part.

Meanwhile, the thigh fixing part 121 and the calf fixing part 123 may have a structure that is connected vertically from the support frame 110 and wrap the leg, and their material may include a plastic or a metal frame with an outer surface having a soft fiber material, rubber or silicone or the like.

Further, the internal side support part and the external support part of the thigh fixing part 121 and the calf fixing part 123 may be configured to wrap and fix a leg according to a leg thickness, using a fix belt, and the fixing belt may include and is configured with a fastening means configured with Velcro tape, a snap button, a button and a buttonhole, a buckle or combination thereof.

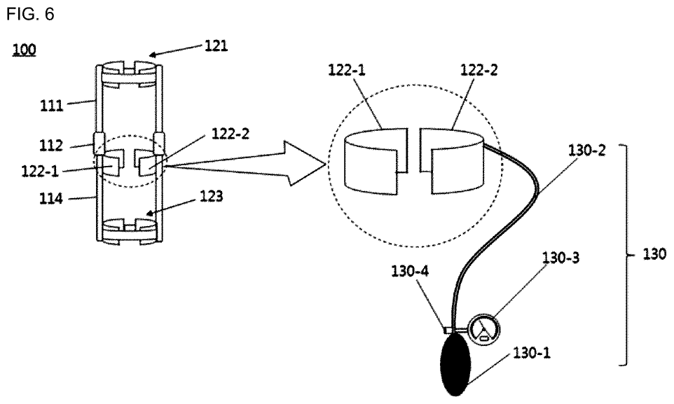

FIGS. 5 and 6 are drawings for explaining about the knee fixing part 122 in the leg correcting apparatus 100 according to an exemplary embodiment of the invention, wherein FIG. 5 is a drawing showing what is about the air cushion part 124 of the knee external side support part 122-2, and FIG. 6 is a drawing illustrating a configuration of injecting, discharging air to or from the air cushion part 124 of the knee external side support part 122-2 and measuring air pressure thereof.

As shown in FIG. 5, the knee external side support part 122-2 in the leg correcting apparatus 100 according to the invention may include the air cushion part 124 for pushing the knee joint portion of the leg antebrachium side from the leg external side toward the leg internal side, and an air supplying part 125 for injecting air to the air cushion part 124.

Further, as shown in FIG. 6, the leg correction apparatus according to the invention may further include an air supplier 130 as a separate apparatus injecting air to the air cushion part.

The air supplier 130 includes and is configured with a pressurizer 130-1 injecting air to the air cushion part of the knee external support part, a tube 130-2 through which air moves from the pressurizer to the air cushion part, an air pressure display part 130-3 displaying air pressure of the air cushion part, and an air vent part 130-4 for removing air pressure of the air cushion part.

That is, by connecting the tube 130-2 to the air supplying part 125 and contracting the pressurizer 130-1, the air cushion part 124 may be supplied with air.

Here, the air cushion part 124 may expand and press the knee joint portion of the leg antebrachium side in the leg internal side direction by the pressure of air injected thereto when air is injected, and by controlling the expansion degree of the air cushion part 124 using the air supplying part 125 and the air vent 130-4, the leg correction may be performed while varying the expansion degree of the air cushion part 124 according to the correction process stage and various user's leg condition.

That is, a user can monitor air pressure in the air cushion part 124 through the air vent part 130-4 of the air supplier 130, and adjust the expansion degree of the air cushion part 124 according to a user's leg condition by adjusting the air pressure of the air cushion 124 while manipulating the pressurizer 130-1 and the air vent part 130-4 referring thereto.

Meanwhile, in FIG. 6 and the above description, the pressurizer 130-1, the tube 130-2, the air pressure display part 130-3 and air vent part 130-4 are described as a configuration included in the air supplier 130 which is a separate apparatus from the knee external side support part 122-2, but the air supplier may be configured to be integral with the leg correcting apparatus 100 by incorporating at least one or more of the pressurizer 130-1, the tube 130-2, the air pressure display part 130-3 and the air vent part 130-4 into the knee external side support part 122-2.

The air pressure display part 130-3, which displays the pressure of air injected into the air cushion part 124, is illustrated as an analogue pressure gauge in FIG. 6, but in reality, may include all analogue pressure gauges or digital pressure gauges capable of displaying the pressure of air injected into the air cushion part 124.

Meanwhile, in the leg correcting apparatus according to the invention, the above description explains that the knee external side support part 122-2 pushes the knee joint portion of the leg antebrachium side from the external side of the leg in the internal side of the leg to apply pressure on the knee external side portion, and that the thigh internal side support part 121-1, the knee internal side support part 122-1 and the calf internal side support part 123-1 support the leg internal side in response to the pressure applied from the knee external side support part 122-2 in the leg internal side direction, whereby the genu varum is corrected, but the invention may correct genu valgum by reversing the configuration of the internal side part and the configuration of the external side part of the leg correcting apparatus 100 in terms of structure and function.

That is, the leg correcting apparatus 100 of the invention may make the knee internal side support part 122-1 push the knee joint portion of the leg antebrachium side from the internal side of the leg in the external side of the leg to apply pressure on the knee internal side portion, and make the thigh external side support part 121-2, the knee external side support part 122-2 and the calf external side support part 123-2 support the leg external side in response to the pressure applied from the knee internal side support part 122-1 in the leg external side direction, whereby the genu valgum is corrected, and as an exemplary configuration of this, the invention may provide a leg correcting apparatus which includes a support frame which can support a brachium and an antebrachium of a leg, and is formed along an up and down direction of the leg; a thigh fixing part which is connected to the support frame and installed so as to wrap and fix a thigh portion of the leg brachium; a calf fixing part which is connected to the support frame and installed so as to wrap and fix a calf portion of the leg antebrachium; and a knee fixing part which is located between the thigh fixing part and the calf fixing part, is connected to the support frame and installed so as to support at least a knee joint portion of the leg antebrachium side, wherein the knee fixing part includes a knee external side support part which is connected and fixed to the support frame, and supports a knee external side; and a knee internal side support part which is connected to the support frame and supports the knee internal side, and which can push the knee joint portion of the leg antebrachium side from the leg internal side in the external side direction to apply a pressure on the knee internal side portion.

While the invention is described with reference to the embodiments shown in the attached drawings, these are just exemplary ones, and those skilled in the art will understand that various modifications or equivalent other embodiments are possible. Accordingly, the technical protection scope of the invention should be determined by the claims below.

### INDUSTRIAL APPLICABILITY

The leg correcting apparatus according to the invention may be widely used in the field of the leg correcting apparatus technology because the tibial externally and laterally deviated can be effectively corrected to a normal position due to a pressure applied in a specific direction only on the knee joint portion of a leg antebrachium side caused by pushing the knee joint portion of the leg antebrachium side from the leg external side in the leg internal side direction to apply pressure on the knee external side portion.

1. A leg correcting apparatus comprising:
a support frame including an upper support frame formed along a leg up and down length direction and supporting a leg brachium, and a lower support frame formed along the leg up and down length direction and supporting a leg antebrachium;
a knee bendable part formed at the upper support frame, the lower support frame or a coupling part thereof and having a structure that can be bent according to a bending angle of a knee;
a thigh fixing part which is connected to the support frame and installed so as to wrap and fix a thigh portion of the leg brachium;
a calf fixing part which is connected to the support frame and installed so as to wrap and fix a calf portion of the leg antebrachium; and
a knee fixing part which is located between the thigh fixing part and the calf fixing part, is connected to the support frame and installed so as to support at least a knee joint portion of the leg antebrachium side,
wherein the knee fixing part includes a knee internal side support part which is connected and fixed to the support frame, and supports a knee internal side, and a knee external side support part which is connected to the support frame and supports the knee external side, and which can push the knee joint portion of the leg antebrachium side from the leg external side in the leg internal side direction to apply a pressure on the knee external side portion.

2. (canceled)

3. The leg correcting apparatus of claim 1, wherein the upper support frame or the lower support frame is provided with one axis at a rear side of a leg, or provided with a pair of axes at the internal side and the external side of the leg brachium.

4. The leg correcting apparatus of claim 1, wherein the knee bendable part is kept unbent by an elastic body including at least one or more selected from a spring, a wire and a rubber if no external force is applied thereto.

5. The leg correcting apparatus of claim 1, wherein the thigh fixing part and the calf fixing part are configured such that their heights can be adjusted along a length direction of a leg when being installed to the support frame, or the support frame is configured such that their lengths can be adjusted along an up and down length direction of a leg.

6. The leg correcting apparatus of claim 1, wherein the thigh fixing part includes a thigh internal side support part which is connected to the support frame and supports a thigh internal side of the leg brachium, and a thigh external side support part which is connected to the support frame and fastened with the thigh internal side support part to support a thigh external side; or the calf fixing part includes a calf internal side support part which is connected to the support frame and supports a calf portion internal side of the leg antebrachium, and a calf external side support part which is connected to the support frame and fastened with the calf internal side support part to support a calf external side.

7. The leg correcting apparatus of claim 6, wherein the thigh internal side support part, the knee internal side support part and the calf internal side support part in the leg correcting apparatus are respectively fixed to the support frame, and are configured to support an internal side of each of the thigh, the knee and the calf in response to a case where pressure is applied to the knee external side portion.

8. The leg correcting apparatus of claim 1, wherein a knee external side support part in the leg correcting apparatus includes an air cushion part which pressurizes the knee joint portion of the leg antebrachium side by pressure of injected air in the internal side direction in order to push the knee joint portion of the leg antebrachium from the leg external side in the leg internal side direction.

9. The leg correcting apparatus of claim 8, further comprising an air supplier as a separate apparatus injecting air to the air cushion part,
wherein the air supplier includes a pressurizer injecting air to the air cushion part of the knee external side support part, and a tube through which air moves from the pressurizer to the air cushion part, an air pressure display part displaying air pressure of the air cushion part, and an air vent part for removing air pressure of the air cushion part.

10. The leg correcting apparatus of claim 1, further comprising an ankle fixing part which is connected to the support frame and installed so as to wrap and support an ankle portion of the leg antebrachium,
wherein the ankle fixing part includes an ankle internal side support part which is connected to the support frame and wraps an ankle portion internal side of the leg antebrachium, and an ankle external side support part which is connected to the support frame and fastened with the ankle internal side support part to support the ankle external side.

11. A leg correcting apparatus comprising:
a support frame including an upper support frame formed along a leg up and down length direction and supporting a leg brachium, and a lower support frame formed along the leg up and down length direction and supporting a leg antebrachium;
a knee bendable part formed at the upper support frame, the lower support frame or a coupling part thereof and having a structure that can be bent according to a bending angle of a knee;
a thigh fixing part which is connected to the support frame and installed so as to wrap and fix a thigh portion of the leg brachium;
a calf fixing part which is connected to the support frame and installed so as to wrap and fix a calf portion of the leg antebrachium; and
a knee fixing part which is located between the thigh fixing part and the calf fixing part, is connected to the support frame and installed so as to support at least a knee joint portion of the leg antebrachium side,
wherein the knee fixing part includes a knee external side support part which is connected and fixed to the support frame, and supports a knee external side, and a knee internal side support part which is connected to the support frame and supports the knee internal side, and which can push the knee joint portion of the leg antebrachium side from the leg internal side in the leg external side direction to apply a pressure on the knee internal side portion.
